(19) United States
(12) Patent Application Publication
(10) Pub. No.: US 2010/0291018 A1
(43) Pub. Date: Nov. 18, 2010

(54) CONDITIONER FOR EYELASH AND EYEBROW

(76) Inventor: Alex KIM, Corona, CA (US)

Correspondence Address: PARK LAW FIRM, 3255 WILSHIRE BLVD, SUITE 1110, LOS ANGELES, CA, 90010, US

(21) Appl. No.: 12/464,826

(22) Filed: May 12, 2009

(51) Int. Cl. A61K 8/97; A61K 8/64; A61Q 5/12

(52) U.S. Cl. 424/74; 424/70.1

(57) ABSTRACT

A conditioner for eyelash and eyebrow includes: water; hydrolyzed soy protein; erythritol; trehalose; carbomer; arginine; polysorbate 80; niacinamide; panthenol; citric acid; phenoxyethanol; acorus calamus root extract; artemisia princes leaf extract; portulaca oleracea extract; polygonum multiflorum root extract; glycyrrhiza glabra root extract; ephedra sinica extract; sophora angustifolia root extract; arctium lappa seed extract; thuja extract; lyceum barbarum fruit extract; ginko biloba leaf extract; paeonia suffruticosa root extract; carthamus tinctorius flower extract; bletilla strita root extract; rheum undulatum root-stalk-stern extract; biotin; methylparaben; and copper tripeptide-1.

### BACKGROUND OF THE INVENTION

The present invention is related to a conditioner for eyelash and eyebrow.

A personal care or cosmetic product for eyelash and eyebrow has been used. Many of prior arts caused irritating or stimulating the eyes.

### SUMMARY OF THE INVENTION

An objective of the invention is to provide a conditioner for eyelash and eyebrow.

An aspect of the invention provides a conditioner for eyelash and eyebrow comprising water, hydrolyzed soy protein, erythritol, trehalose, carbomer, arginine, polysorbate 80, niacinamide, panthenol, citric acid, and phenoxyethanol.

The conditioner may comprise water from about 94.0 percent to about 97 percent. In certain embodiments of the invention, the conditioner may comprise water of about 95.68 percent.

The conditioner may comprise hydrolyzed soy protein from about 0.6 percent to about 0.7 percent. In certain embodiments of the invention, the conditioner may comprise hydrolyzed soy protein of about 0.65 percent.

The conditioner may comprise erythritol from about 0.55 percent to about 0.65 percent. In certain embodiments of the invention, the conditioner may comprise erythritol of about 0.60 percent.

The conditioner may comprise trehalose from about 0.55 percent to about 0.65 percent. In certain embodiments of the invention, the conditioner may comprise trehalose from about 0.60 percent.

The conditioner may comprise carbomer from about 0.54 percent to about 0.64 percent. In certain embodiments of the invention, the conditioner of claim 10, wherein the conditioner may comprise carbomer from about 0.59 percent.

The conditioner may comprise: arginine from about 0.16 percent to about 0.26 percent; polysorbate 80 from about 0.15 percent to about 0.25 percent; niacinamide from about 0.15 percent to about 0.25 percent; panthenol from about 0.10 percent to about 0.20 percent; citric acid from about 0.08 percent to about 0.18 percent; and phenoxyethanol from about 0.05 percent to about 0.15 percent.

In certain embodiments of the invention, the conditioner may comprise: arginine of about 0.21 percent; polysorbate 80 of about 0.20 percent; niacinamide of about 0.20 percent; panthenol of about 0.15 percent; citric acid of about 0.13 percent; and phenoxyethanol of about 0.10 percent.

The conditioner may further comprise: acorus calamus root extract; artemisia princes leaf extract; portulaca oleracea extract; polygonum multiflorum root extract; glycyrrhiza glabra root extract; ephedra sinica extract; sophora angustifolia root extract; arctium lappa seed extract; and thuja extract.

In certain embodiments of the invention, the conditioner of claim 14, wherein the conditioner may comprise: acorus calamus root extract of about 0.05 percent; artemisia princes leaf extract of about 0.05 percent; portulaca oleracea extract of about 0.05 percent; polygonum multiflorum root extract of about 0.05 percent; glycyrrhiza glabra root extract of about 0.05 percent; ephedra sinica extract of about 0.05 percent; sophora angustifolia root extract of about 0.05 percent; arctium lappa seed extract of about 0.05 percent; and thuja extract of about 0.05 percent.

In certain embodiments of the invention, the conditioner may further comprise: lyceum barbarum fruit extract; ginko biloba leaf extract; paeonia suffruticosa root extract; carthamus tinctorius flower extract; bletilla strita root extract; rheum undulatum root-stalk-stern extract; biotin; methylparaben; and copper tripeptide-1.

Preferably, the conditioner may comprise: lyceum barbarum fruit extract of about 0.05 percent; ginko biloba leaf extract of about 0.05 percent; paeonia suffruticosa root extract of about 0.05 percent; carthamus tinctorius flower extract of about 0.05 percent; bletilla strita root extract of about 0.05 percent; rheum undulatum root-stalk-stern extract of about 0.05 percent; biotin of about 0.05 percent; methylparaben of about 0.05 percent; and copper tripeptide-1 of about 0.04 percent.

Another aspect of the invention provides a conditioner for eyelash and eyebrow comprising: water of about 95.68 percent; hydrolyzed soy protein of about 0.65 percent; erythritol of about 0.60 percent; trehalose of about 0.60 percent; carbomer of about 0.59 percent; arginine of about 0.21 percent; polysorbate 80 of about 0.20 percent; niacinamide of about 0.20 percent; panthenol of about 0.15 percent; citric acid of about 0.13 percent; phenoxyethanol of about 0.10 percent; acorus calamus root extract of about 0.05 percent; artemisia princes leaf extract of about 0.05 percent; portulaca oleracea extract of about 0.05 percent; polygonum multiflorum root extract of about 0.05 percent; glycyrrhiza glabra root extract of about 0.05 percent; ephedra sinica extract of about 0.05 percent; sophora angustifolia root extract of about 0.05 percent; arctium lappa seed extract of about 0.05 percent; thuja extract of about 0.05 percent; lyceum barbarum fruit extract of about 0.05 percent; ginko biloba leaf extract of about 0.05 percent; paeonia suffruticosa root extract of about 0.05 percent; carthamus tinctorius flower extract of about 0.05 percent; bletilla strita root extract of about 0.05 percent; rheum undulatum root-stalk-stern extract of about 0.05 percent; biotin of about 0.05 percent; methylparaben of about 0.05 percent; and copper tripeptide-1 of about 0.04 percent.

The advantages of the present invention are: (1) the conditioner is for eyelash and eyebrow; and (2) the conditioner is skin-friendly.

Although the present invention is briefly summarized, the fuller understanding of the invention can be obtained by the following drawings, detailed description and appended claims.

### DETAILED DESCRIPTION OF THE INVENTION

An aspect of the invention provides a conditioner for eyelash and eyebrow comprising water, hydrolyzed soy protein, erythritol, trehalose, carbomer, arginine, polysorbate 80, niacinamide, panthenol, citric acid, and phenoxyethanol.

In order to obtain desired effects, the above ingredients must be in included in the conditioner by a specific amount. CAS numbers for the ingredients are given in parentheses after name of the corresponding ingredient.

The conditioner may comprise water (7732-15-8) from about 94.0 percent to about 97 percent. In certain embodiments of the invention, the conditioner may comprise water of about 95.68 percent.

The conditioner may comprise hydrolyzed soy protein from about 0.6 percent to about 0.7 percent. In certain embodiments of the invention, the conditioner may comprise hydrolyzed soy protein of about 0.65 percent.

The conditioner may comprise erythritol (149-32-6) from about 0.55 percent to about 0.65 percent. In certain embodiments of the invention, the conditioner may comprise erythritol of about 0.60 percent.

The conditioner may comprise trehalose (99-20-7) from about 0.55 percent to about 0.65 percent. In certain embodiments of the invention, the conditioner may comprise trehalose from about 0.60 percent.

The conditioner may comprise carbomer (9003-01-4) from about 0.54 percent to about 0.64 percent. In certain embodiments of the invention, the conditioner of claim 10, wherein the conditioner may comprise carbomer from about 0.59 percent.

The conditioner may comprise: arginine (74-79-3) from about 0.16 percent to about 0.26 percent; polysorbate 80 (9005-65-6) from about 0.15 percent to about 0.25 percent; niacinamide (98-92-0) from about 0.15 percent to about 0.25 percent; panthenol (16485-10-2) from about 0.10 percent to about 0.20 percent; citric acid (77-92-9) from about 0.08 percent to about 0.18 percent; and phenoxyethanol (122-99-6) from about 0.05 percent to about 0.15 percent.

In certain embodiments of the invention, the conditioner may comprise: arginine of about 0.21 percent; polysorbate 80 of about 0.20 percent; niacinamide of about 0.20 percent; panthenol of about 0.15 percent; citric acid of about 0.13 percent; and phenoxyethanol of about 0.10 percent.

The conditioner may further comprise: acorus calamus root extract; artemisia princes leaf extract; portulaca oleracea extract; polygonum multiflorum root extract; glycyrrhiza glabra root extract; ephedra sinica extract; sophora angustifolia root extract; arctium lappa seed extract; and thuja extract.

In certain embodiments of the invention, the conditioner may comprise: acorus calamus root extract (84775-39-3) of about 0.05 percent; artemisia princes leaf extract of about 0.05 percent; portulaca oleracea extract (90083-07-1) of about 0.05 percent; polygonum multiflorum root extract of about 0.05 percent; glycyrrhiza glabra root extract (84775-66-6) of about 0.05 percent; ephedra sinica extract (85940-38-1) of about 0.05 percent; sophora angustifolia root extract of about 0.05 percent; arctium lappa seed extract (84012-13-5) of about 0.05 percent; and thuja extract of about 0.05 percent.

In certain embodiments of the invention, the conditioner may further comprise: lyceum barbarum fruit extract; ginko biloba leaf extract; paeonia suffruticosa root extract; carthamus tinctorius flower extract; bletilla strita root extract; rheum undulatum root-stalk-stern extract; biotin; methylparaben; and copper tripeptide-1.

Preferably, the conditioner may comprise: lyceum barbarum fruit extract of about 0.05 percent; ginko biloba leaf extract (90045-36-6) of about 0.05 percent; paeonia suffruticosa root extract of about 0.05 percent; carthamus tinctorius flower extract of about 0.05 percent; bletilla strita root extract of about 0.05 percent; rheum undulatum root-stalk-stern extract (8016-55-5) of about 0.05 percent; biotin (58-85-5) of about 0.05 percent; methylparaben (99-76-3) of about 0.05 percent; and copper tripeptide-1 of about 0.04 percent.

Another aspect of the invention provides a conditioner for eyelash and eyebrow comprising: water of about 95.68 percent; hydrolyzed soy protein of about 0.65 percent; erythritol of about 0.60 percent; trehalose of about 0.60 percent; carbomer of about 0.59 percent; arginine of about 0.21 percent; polysorbate 80 of about 0.20 percent; niacinamide of about 0.20 percent; panthenol of about 0.15 percent; citric acid of about 0.13 percent; phenoxyethanol of about 0.10 percent; acorus calamus root extract of about 0.05 percent; artemisia princes leaf extract of about 0.05 percent; portulaca oleracea extract of about 0.05 percent; polygonum multiflorum root extract of about 0.05 percent; glycyrrhiza glabra root extract of about 0.05 percent; ephedra sinica extract of about 0.05 percent; sophora angustifolia root extract of about 0.05 percent; arctium lappa seed extract of about 0.05 percent; thuja extract of about 0.05 percent; lyceum barbarum fruit extract of about 0.05 percent; ginko biloba leaf extract of about 0.05 percent; paeonia suffruticosa root extract of about 0.05 percent; carthamus tinctorius flower extract of about 0.05 percent; bletilla strita root extract of about 0.05 percent; rheum undulatum root-stalk-stern extract of about 0.05 percent; biotin of about 0.05 percent; methylparaben of about 0.05 percent; and copper tripeptide-1 of about 0.04 percent.

The conditioner is provided liquid, and has color of clear and pale yellow, but does not have any odor.

The conditioner is used on clean skin and with clean hands. None of the ingredients in the conditioner is listed as carcinogens in NTP, IARC Monographs, or OSHA Regulated carcinogens.

While the invention has been shown and described with reference to different embodiments thereof, it will be appreciated by those skilled in the art that variations in form, detail, compositions and operation may be made without departing from the spirit and scope of the invention as defined by the accompanying claims. For example, the rotating guide surface 211 may be formed convex and the rotation surface 111 may be formed concave.

What is claimed is:

1. A conditioner for eyelash and eyebrow comprising:
water;
hydrolyzed soy protein;
erythritol;
trehalose;
carbomer;
arginine;
polysorbate 80;
niacinamide;
panthenol;
citric acid; and
phenoxyethanol.

2. The conditioner of claim 1, wherein the conditioner comprises water from about 94.0 percent to about 97 percent.

3. The conditioner of claim 2, wherein the conditioner comprises water of about 95.68 percent.

4. The conditioner of claim 1, wherein the conditioner comprises hydrolyzed soy protein from about 0.6 percent to about 0.7 percent.

5. The conditioner of claim 4, wherein the conditioner comprises hydrolyzed soy protein of about 0.65 percent.

6. The conditioner of claim 1, wherein the conditioner comprises erythritol from about 0.55 percent to about 0.65 percent.

7. The conditioner of claim 6, wherein the conditioner comprises erythritol of about 0.60 percent.

8. The conditioner of claim 1, wherein the conditioner comprises trehalose from about 0.55 percent to about 0.65 percent.

9. The conditioner of claim 8, wherein the conditioner comprises trehalose from about 0.60 percent.

10. The conditioner of claim 1, wherein the conditioner comprises carbomer from about 0.54 percent to about 0.64 percent.

11. The conditioner of claim 10, wherein the conditioner comprises carbomer from about 0.59 percent.

12. The conditioner of claim 1, wherein the conditioner comprises:
arginine from about 0.16 percent to about 0.26 percent;
polysorbate 80 from about 0.15 percent to about 0.25 percent;
niacinamide from about 0.15 percent to about 0.25 percent;
panthenol from about 0.10 percent to about 0.20 percent;
citric acid from about 0.08 percent to about 0.18 percent; and
phenoxyethanol from about 0.05 percent to about 0.15 percent.

13. The conditioner of claim 12, wherein the conditioner comprises:
arginine of about 0.21 percent;
polysorbate 80 of about 0.20 percent;
niacinamide of about 0.20 percent;
panthenol of about 0.15 percent;
citric acid of about 0.13 percent; and
phenoxyethanol of about 0.10 percent.

14. The conditioner of claim 1, further comprising:
acorus calamus root extract;
artemisia princes leaf extract;
portulaca oleracea extract;
polygonum multiflorum root extract;
glycyrrhiza glabra root extract;
ephedra sinica extract;
sophora angustifolia root extract;
arctium lappa seed extract; and
thuja extract.

15. The conditioner of claim 14, wherein the conditioner comprises:
acorus calamus root extract of about 0.05 percent;
artemisia princes leaf extract of about 0.05 percent;
portulaca oleracea extract of about 0.05 percent;
polygonum multiflorum root extract of about 0.05 percent;
glycyrrhiza glabra root extract of about 0.05 percent;
ephedra sinica extract of about 0.05 percent;
sophora angustifolia root extract of about 0.05 percent;
arctium lappa seed extract of about 0.05 percent; and
thuja extract of about 0.05 percent.

16. The conditioner of claim 14, further comprising:
lyceum barbarum fruit extract;
ginko biloba leaf extract;
paeonia suffruticosa root extract;
carthamus tinctorius flower extract;
bletilla strita root extract;
rheum undulatum root-stalk-stern extract;
biotin;
methylparaben; and
copper tripeptide-1.

17. The conditioner of claim 16, wherein the conditioner comprises:
lyceum barbarum fruit extract of about 0.05 percent;
ginko biloba leaf extract of about 0.05 percent;
paeonia suffruticosa root extract of about 0.05 percent;
carthamus tinctorius flower extract of about 0.05 percent;
bletilla strita root extract of about 0.05 percent;
rheum undulatum root-stalk-stern extract of about 0.05 percent;
biotin of about 0.05 percent;
methylparaben of about 0.05 percent; and
copper tripeptide-1 of about 0.04 percent.

18. A conditioner for eyelash and eyebrow comprising:
Water of about 95.68 percent;
hydrolyzed soy protein of about 0.65 percent;
erythritol of about 0.60 percent;
trehalose of about 0.60 percent;
carbomer of about 0.59 percent;
arginine of about 0.21 percent;
polysorbate 80 of about 0.20 percent;
niacinamide of about 0.20 percent;
panthenol of about 0.15 percent;
citric acid of about 0.13 percent;
phenoxyethanol of about 0.10 percent;
acorus calamus root extract of about 0.05 percent;
artemisia princes leaf extract of about 0.05 percent;
portulaca oleracea extract of about 0.05 percent;
polygonum multiflorum root extract of about 0.05 percent;
glycyrrhiza glabra root extract of about 0.05 percent;
ephedra sinica extract of about 0.05 percent;
sophora angustifolia root extract of about 0.05 percent;
arctium lappa seed extract of about 0.05 percent;
thuja extract of about 0.05 percent;
lyceum barbarum fruit extract of about 0.05 percent;
ginko biloba leaf extract of about 0.05 percent;
paeonia suffruticosa root extract of about 0.05 percent;
carthamus tinctorius flower extract of about 0.05 percent;
bletilla strita root extract of about 0.05 percent;
rheum undulatum root-stalk-stern extract of about 0.05 percent;
biotin of about 0.05 percent;
methylparaben of about 0.05 percent; and
copper tripeptide-1 of about 0.04 percent.
